(19) United States
(12) Patent Application Publication
(10) Pub. No.: US 2006/0200182 A1
(43) Pub. Date: Sep. 7, 2006

(54) SAFETY SHIELDED TROCAR WITH OBLIQUE INCISION EDGES

(75) Inventor: Michael Prosek, Jacksonville, FL (US)

Correspondence Address: Mark James Young, 12086 Fort Caroline Rd., Unit 202, Jacksonville, FL, 32225, US

(73) Assignee: LAPAROSCOPIC PARTNERS LLC, Jacksonville, FL (US)

(21) Appl. No.: 11/308,066

(22) Filed: Mar. 5, 2006

Related U.S. Application Data
(60) Provisional application No. 60/658,765, filed on Mar. 5, 2005.

(51) Int. Cl. A61B 17/34

(52) U.S. Cl. 606/185

(57) ABSTRACT

A trocar includes an incision blade/safety shield cartridge assembly, a sleeve body having a distal end and an opposite proximal end, and a handle. The incision blade/safety shield cartridge assembly includes a nose cone; a stationary incision blade adapted to produce a clean incision with a flap, such as a v-shaped incision; and a retractable safety shield that conforms to the shape of the incision blade, a spring, and a safety shield spring retainer housing. The safety shield retracts to a retracted position exposing the incision blade during piercing of a body cavity and rapidly extends to an extended position beyond the tip of the incision blade upon penetration of a body cavity, thereby guarding against unintended incision and/or puncture wounds.

### RELATED APPLICATION

This application claims the benefit of priority of U.S. Provisional Application 60/658,765, filed Mar. 5, 2005, the entire contents of which are incorporated herein.

### FIELD OF THE INVENTION

This invention relates generally to a surgical instrument, and, more specifically, to a safety shielded trocar having a V-shaped incision blade.

### BACKGROUND

In laparoscopic surgery, a surgeon guides cameras and long, thin instruments through small incisions in the body. As laparoscopy is less invasive than conventional surgery, laparoscopic techniques typically result in quicker, less painful operations, with less scarring and shorter recovery times. Today, examples of laparoscopic procedures abound. They include tubal ligations, hysterectomies, surgery for endometriosis or other common gynecological problems, gall bladder surgery, and some hernia and heart operations.

A surgeon typically starts a laparoscopic operation by injecting carbon dioxide gas into a patient's abdomen through a thin needle to create more space between the abdominal wall and the organs. Then, the surgeon makes a piercing incision, using a razor-sharp instrument called a trocar. A conventional trocar is essentially a metal spike contained within a spring-loaded safety sheath. The tip of the trocar is typically needle-like with a beveled piercing tip having sharp edges. The spring-loaded safety sheath is a retractable sleeve positioned around the trocar. The sheath slides back upon contact with the outer surface and walls of the body cavity to reveal a sharp incision edge. After the internal cavity has been breached, the sheath springs forward to cover the sharp incision edge.

Another style of trocar features a sharp tubular needle with an internal blunt spring-biased obturator. The obturator retracts into the body of the needle during piercing and blocks the interior of the needle to prevent tissue from entering. When the tip of the trocar enters the insufflated cavity, the biasing spring pushes the obturator forward past the sharp tip of the sleeve to prevent accidental puncturing or cutting of internal organs.

To make an incision with a trocar, a surgeon pushes a trocar through a sealable cannula, skin, fat and connective tissue and into the abdominal cavity. Since the surgeon has not yet inserted a camera, the surgeon cannot see the sharp trocar as it penetrates the body cavity, which may lead to serious collateral injuries. Once the trocar is inserted, the protective spring-loaded sheath should spring forward, covering the blade and protecting arteries and organs. However, the sheath (or obturator) may not always deploy fast enough due to interfering tissue or other mechanical interference. The sheath (or obturator) may also become caught (or plugged up) on tissue and fail to deploy. Additionally, if a surgeon pushes too hard, the force may break the spring. While some manufacturer's labels may warn against pushing too hard, there is no way of gauging how hard is too hard. Compounding these problems, is the fact that safety-shielded trocars might actually lead to accidents because they give surgeons a false sense of security, encouraging them to use more force.

The only indication of penetration provided by a standard trocar is a reduction in the amount of resistance felt by the surgeon. Consequently, it can be extremely difficult for a surgeon to ascertain when the internal cavity wall has been breached. To address this problem, visible and audible signaling devices have been developed to provide a positive signal when a cavity wall has been breached. However, if the signal is missed or the surgeon fails to react in time, the result can be serious collateral damage.

After the trocar has been driven into the body cavity, the surgeon may withdraw it and proceed with the laporascopic procedure. The result, when no blood vessels or organs are cut, is quick and easy access to the abdomen. Upon withdrawal of the trocar, a cannula is left in place to provide a sealable access conduit to the insufflated body cavity.

Another problem with conventional trocars concerns the shape of the cutting blade. Straight line incisions have a tendency to tear and result in greater trauma to neighboring areas, especially upon insertion of a laparoscopic instrument. T-shaped and Y-shaped cutting blades require greater force to pierce the cavity, thereby producing more trauma and scarring.

Other problems with such trocar assemblies include the capture of tissue intermediate the obturator and the piercing sleeve wall when the obturator is retracted or pushed back by the body cavity wall. Since the tip is beveled, the initial piercing and cutting is performed by the leading edge of the blade formed on the beveled edge of the piercing sleeve. An opening at the trailing edge of the beveled tip is not as smoothly formed as the initial cut, and retraction of the obturator can capture tissue intermediate the obturator and sleeve.

Although attempts have been made to provide a trocar which facilitates penetration, minimizes tearing and trauma, reliably guards against collateral damage upon insertion and provides a clear positive penetration signal, known trocars provided to date have failed to address this full range of surgeons' needs.

The invention is directed to fulfilling one or more of the needs and overcoming one or more of the problems as set forth above.

### SUMMARY OF THE INVENTION

To overcome one or more of the problems as set forth above, in one aspect of the invention, a surgical instrument comprised of an incision blade/safety shield cartridge assembly is provided. The incision blade/safety shield cartridge assembly is comprised of a nose cone having an aperture, a stationary V-shaped incision blade having a distal end extending from said aperture, a spring-biased V-shaped safety shield movable from an extended position extending beyond the distal end of said stationary V-shaped incision blade to a retracted position revealing the distal end of said stationary V-shaped incision blade. The V-shaped safety shield has a distal end and a proximal end. A spring is adapted to bias the spring-biased V-shaped safety shield. A safety shield spring retainer housing is operably coupled to the nose cone and configured to support the spring against the proximal end of the spring-biased V-shaped safety shield.

In an exemplary embodiment, the spring-biased V-shaped safety shield includes a travel stop, and the safety shield spring retainer housing includes a stop lug. The stop lug is adapted to define an abutment for the travel stop when the spring-biased V-shaped safety shield is biased to the extended position. The travel stop and stop lug may also be adapted to produce a sensible signal when the spring-biased V-shaped safety shield is biased to the extended position. The sensible signal may include tactile, audible and visible signals.

A surgical instrument according to an exemplary embodiment is also comprised of, a sleeve body having a distal end and an opposite proximal end, and a handle. The incision blade/safety shield cartridge assembly is coupled to the sleeve body at the distal end, the handle is coupled to the sleeve body at the proximal end.

Thus, in one embodiment, an incision blade/safety shield cartridge assembly according to principles of the invention includes a nose cone having an aperture, a stationary V-shaped incision blade having a distal end extending from the aperture, and a spring-biased V-shaped safety shield conformed to the shape of the stationary V-shaped incision blade and movable from an extended position extending beyond the distal end of the stationary V-shaped incision blade to a retracted position revealing the distal end of the stationary V-shaped incision blade. The V-shaped safety shield has a distal end and a proximal end. A spring is adapted to bias the spring-biased V-shaped safety shield. A safety shield spring retainer housing is operably coupled to the nose cone and configured to support the spring against the proximal end of the spring-biased V-shaped safety shield. The spring-biased V-shaped safety shield may include a travel stop and the safety shield spring retainer housing may include a stop lug adapted to define an abutment for the travel stop when the spring-biased V-shaped safety shield is biased to the extended position. Additionally, the travel stop and stop lug may be adapted to produce a sensible signal when the spring-biased V-shaped safety shield is biased to the extended position. The sensible signal may include a tactile signal, an audible signal or a visible signal. The nose cone may includes a biocompatible lubricated surface treatment. The V-shaped incision blade is adapted with a pointed double-beveled edge at its distal end to produce a clean v-shaped incision in a body cavity. In operation, the V-shaped safety shield retracts to the retracted position during piercing of a body cavity and rapidly extends to the extended position upon penetration of a body cavity.

In another embodiment, a surgical instrument according to principles of the invention includes an incision blade/safety shield cartridge assembly, a sleeve body having a distal end and an opposite proximal end, and a handle. The incision blade/safety shield cartridge is coupled to the sleeve body at the distal end. The handle is coupled to the sleeve body at the proximal end. The incision blade/safety shield cartridge assembly is comprised of a nose cone having an aperture, a stationary V-shaped incision blade having a distal end extending from the aperture, a spring-biased V-shaped safety shield conformed to the shape of the stationary V-shaped incision blade and movable from an extended position extending beyond the distal end of the stationary V-shaped incision blade to a retracted position revealing the distal end of the stationary V-shaped incision blade. The V-shaped safety shield has a distal end and a proximal end, a spring adapted to bias the spring-biased V-shaped safety shield, and a safety shield spring retainer housing operably coupled to the nose cone and configured to support the spring against the proximal end of the spring-biased V-shaped safety shield. The handle has a mushroom shape.

The nose cone includes a biocompatible lubricated surface treatment. The V-shaped incision blade includes a pointed double-beveled edge at its distal end adapted to produce a clean v-shaped incision. The V-shaped safety shield is adapted to retract to the retracted position during piercing of a body cavity and rapidly extend to the extended position upon penetration of a body cavity. Upon such extension, the safety shield produces a sensible tactile, audible and/or visible signal.

In yet another embodiment, a surgical instrument according to principles of the invention includes an incision blade/safety shield cartridge assembly, a sleeve body having a distal end and an opposite proximal end, and a handle. The incision blade/safety shield cartridge assembly is coupled to the sleeve body at the distal end. The handle is coupled to the sleeve body at the proximal end. The incision blade/safety shield cartridge assembly is comprised of a nose cone having an aperture, a stationary incision blade with a distal end extending from the aperture, a spring-biased safety shield conformed to the shape of the stationary incision blade and movable from an extended position extending beyond the distal end of the stationary incision blade to a retracted position revealing the distal end of the stationary incision blade. The safety shield has a distal end and a proximal end, a spring adapted to bias the spring-biased safety shield, and a safety shield spring retainer housing operably coupled to the nose cone and configured to support the spring against the proximal end of the spring-biased safety shield. The safety shield is adapted to retract to the retracted position during piercing of a body cavity and rapidly extend to the extended position upon penetration of a body cavity and produce a sensible signal such as a tactile signal, an audible signal and/or a visible signal. The incision blade includes a pointed double-beveled edge at its distal end is adapted to produce a clean incision with a flap.

### BRIEF DESCRIPTION OF THE DRAWINGS

The foregoing and other aspects, objects, features and advantages of the invention will become better understood with reference to the following description, appended claims, and accompanying drawings, where:

FIG. 1 illustrates an exterior view of an exemplary trocar according to principles of the invention;

FIG. 2 illustrates an exterior view of an exemplary trocar according to principles of the invention with a sleeve adapter at the distal end;

FIG. 2A illustrates an exterior view of an exemplary sleeve adapter according to principles of the invention;

FIG. 3 illustrates a section view of an exemplary trocar according to principles of the invention;

FIG. 4 illustrates an exploded top view of an exemplary trocar according to principles of the invention;

FIG. 5 illustrates an exploded 1 80 degree bottom view of an exemplary trocar according to principles of the invention;

FIG. 6 illustrates a section view of an exemplary safety-shield cartridge assembly with an incision blade in shielded position for a trocar according to principles of the invention;

FIG. 6A illustrates a top solid view of an exemplary safety-shield cartridge assembly with an incision blade in shielded position for a trocar according to principles of the invention;

FIG. 6B illustrates a bottom solid view of an exemplary safety-shield cartridge assembly with an incision blade in shielded position for a trocar according to principles of the invention;

FIG. 6C illustrates an piercing end view of an exemplary safety-shield cartridge assembly with an incision blade in shielded position for a trocar according to principles of the invention;

FIG. 7 illustrates a section view of an exemplary safety-shield cartridge assembly with an incision blade in exposed position for a trocar according to principles of the invention;

FIG. 7A illustrates a solid top view of an exemplary safety-shield cartridge assembly with an incision blade in exposed position for a trocar according to principles of the invention;

FIG. 7B illustrates a solid bottom view of an exemplary safety-shield cartridge assembly with an incision blade in exposed position for a trocar according to principles of the invention; and

FIG. 8 illustrates an exemplary safety-shielded trocar installed into an exemplary cannula with a seal cap assembly for piercing a body cavity wall according to principles of the invention; and

FIG. 9 illustrates a side perspective view of an exemplary v-shaped incision blade and a correspondingly shaped retractable safety shield for piercing a body cavity wall according to principles of the invention; and

FIG. 10 illustrates a side perspective view of an exemplary v-shaped incision blade and a correspondingly shaped retractable safety shield for piercing a body cavity wall according to principles of the invention; and

FIG. 11 illustrates a tip-end plan view of an exemplary retractable safety shield according to principles of the invention; and

FIG. 12 illustrates a tip-end plan view of an exemplary v-shaped incision blade for piercing a body cavity wall according to principles of the invention.

Those skilled in the art will appreciate that the invention is not limited to the exemplary embodiments depicted in the figures or the shapes, relative sizes, proportions or materials shown in the figures.

### DETAILED DESCRIPTION

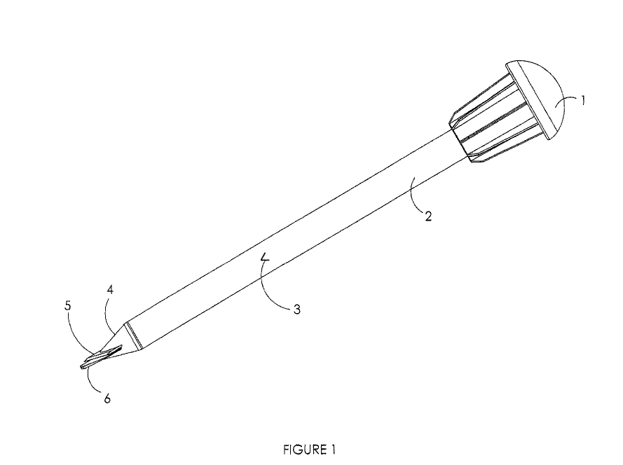

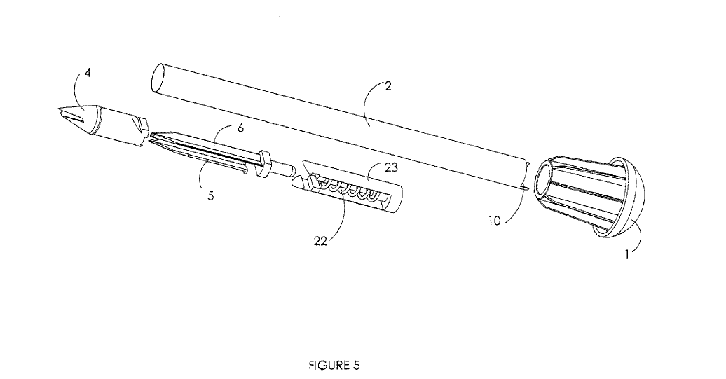

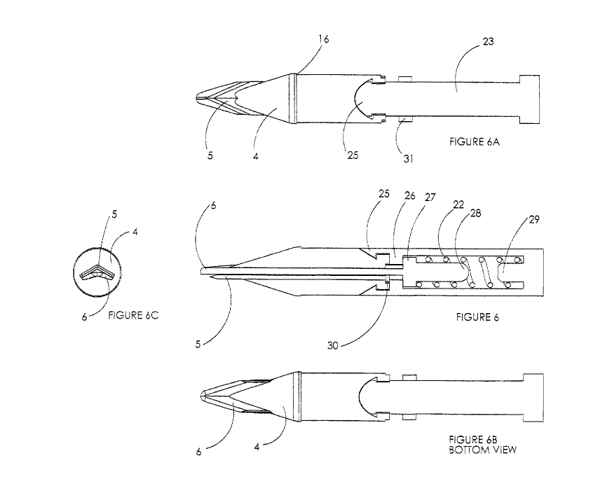

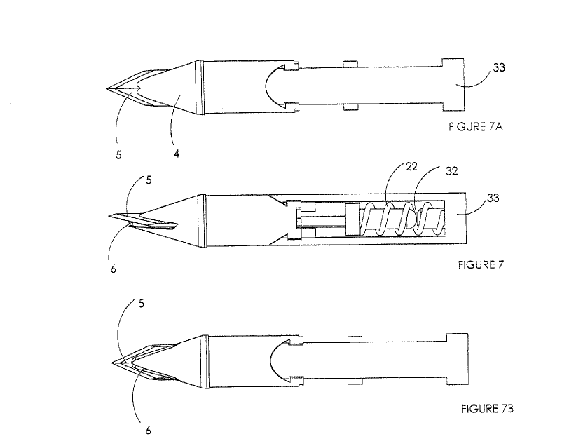

With reference to the drawings, wherein like numerals represent like features, an exemplary trocar and components thereof according to principles of the invention are shown in FIGS. 1 through 12. The invention provides an improved trocar for safely creating an incision in a patient (either human or animal) and establishing an orifice for laparoscopic medical procedures.

Referring now to FIG. 1, an exterior perspective view of exemplary embodiment of a trocar according to principles of the invention is illustrated. As shown, the trocar includes a handle 1, a sleeve body 2, a staking detail 3, a nose cone 4, an incision blade 5 and a retractable safety shield 6. The incision blade 5, retractable safety shield 6, and other components(as discussed below) are housed or partially housed within the nose cone 4 and sleeve body 2. An incision blade/safety shield cartridge assembly (discussed below) is fixed at the distal end using the indented staking detail 3. The nose cone 4 includes an aperture for passage of the sharpened distal end of the incision blade 5 and safety shield 6. The nose cone 4 and handle 1 are attached to the sleeve body 2 in any conventional manner, such as by bonding or mechanical connection. For surgeon comfort and control, the handle 1 preferably has a mushroom shape, though other shapes may be used.

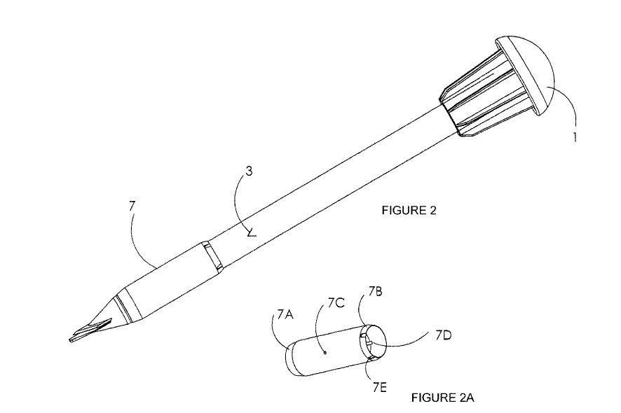

Optionally, a sleeve adapter 7 is provided, as shown in FIG. 2. The sleeve adapter 7 may be attached (e.g., bonded) as a collar onto the sleeve tube/body member 2 for the purpose of increasing the effective outer diameter of the sleeve tube/body member 2. Illustratively, the sleeve adapter 7 may be configured to increase the effective outer diameter of the sleeve tube/body member 2 from 10 mm diameter to 12 mm diameter for use in a cannula with a 12 mm inner diameter. By way of example and not limitation, the sleeve adapter 7 may be comprised plastic of polyethylene, polycarbonate, ABS or other plastic resin.

Now turning to FIG. 2A, the exemplary sleeve adapter 7 includes a conical taper 7A at the distal end to continue the conical taper of the nose cone 4 for smooth and gradual incision dilation. A conical taper 7B is also provided at the proximal end to facilitate removal of the trocar from a cannula after the puncturing. The conical taper 7B at the proximal end also reduces the possibility of rupturing the rubber gas seal typically contained within a cannula. A glue injection port 7C is provided for injecting glue utilizing a mating glue injection needle (not shown) to bond the sleeve adapter 7 into a predetermined fixed position onto the sleeve tube/body member 2. Internal standing ribs 7D of a determined number and height may also be provided to assure good distribution and adequate glue film thickness between the sleeve adapter 7 and the sleeve tube/body member 2. Detents 7E may be provided to facilitate ejection of the part from the plastic injection mold.

With reference to FIG. 3A, an exemplary trocar according to the present invention includes a substantially hollow sleeve tube/body member 2 with an interior cavity 13 and a tubular wall. The distal end of hollow sleeve tube/body member 2 engages an incision blade/safety shield cartridge assembly 8. By way of example and not limitation, the sleeve tube/body member 2 is made from thin wall stainless steel tubing of polished or mat finish, or of polycarbonate, ABS or other plastic resin, or any other material suitable for surgical applications.

The distal end, receives the incision blade/safety shield cartridge assembly 8 to a determined depth. The cartridge 8 may be fixed at the distal end with one or more attachments, such as a “V” shaped indented staking detail 3, as shown in FIG. 1. The “V” shaped indented staking detail 3 is used for fixed retention of the internal incision blade/safety-shield cartridge assembly. One or a plurality of spaced apart staking details 3 may be provided to secure the cartridge to the sleeve tube/body member 2.

The handle 1 may be fixed by bonding or mechanical attachment onto the proximal end of the sleeve tube/body member 2. An open chamber or bore 12 in the handle 1 receives a portion of the proximal end of the sleeve tube/body member 2. The handle 1 may be attached to the proximal end of the sleeve tube/body member 2 by bonding or other suitable fastening means.

As illustrated in FIGS. 3, the bore 12 of the handle incorporates a standing tube stop lug 9 near the bottom of the bore 12 of about twenty-five percent of the circumference of the bore 12 and standing inwardly approximately twice the thickness of the wall thickness of the sleeve tube/body member 2. The standing tube stop lug 9 with recess 9A and plateau 9B in the bore 12 is configured to selectively mate with a corresponding tube notch 10A or plateau 10B of the proximal end of the sleeve tube/body member 2. The height of the stop lug 9 from the bottom of the bore 12 is the same or slightly less than the depth of the tube notch depth. A long configuration is achieved when plateau 9B aligns with plateau 10B. A short configuration is achieved when recess 9A is aligned with plateau 10B. The sleeve tube/body member 2 may be manually rotated to an appropriate position to achieve a determined length, i.e., either a long version or short version. Thus, advantageously, the tube stop lug 9 with recess 9A and plateau 9B and corresponding tube notch 10A and plateau 10B enable selection of a first or second length of the trocar, to accommodate various cannulas and laparoscopic procedures.

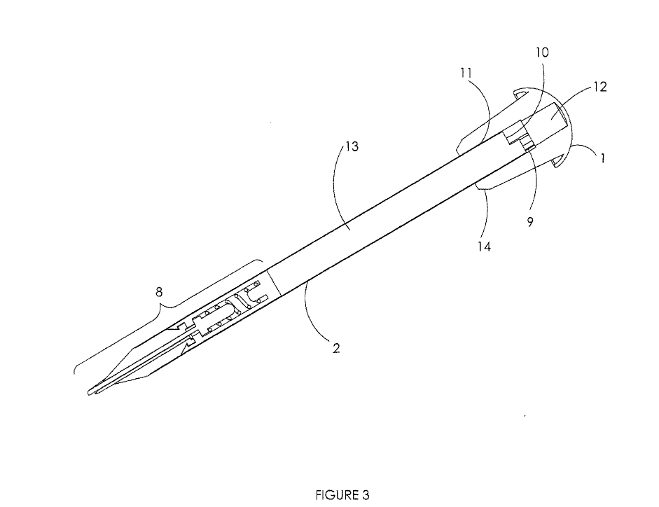

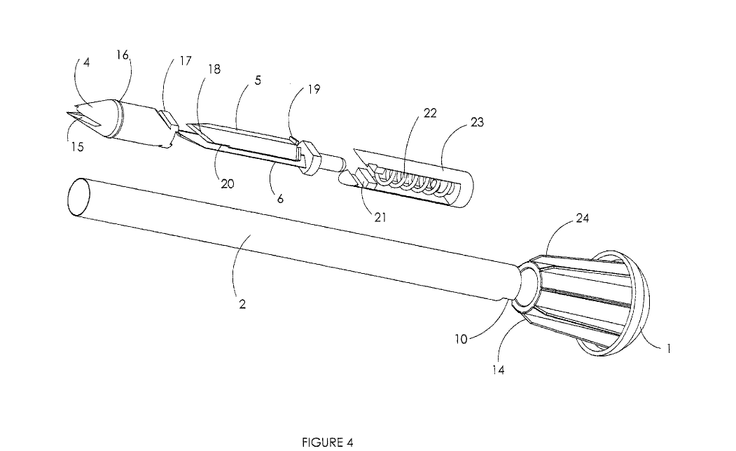

Referring now to FIG. 4, an exploded top view of an exemplary trocar and incision blade/safety shield cartridge assembly 8 according to principles of the invention is shown. The incision blade/safety shield cartridge assembly 8 includes a nose cone 4 with an aperture 15 for passage of the distal ends of an incision blade 5 and a correspondingly shaped retractable safety shield 6. In an exemplary implementation, the incision blade 5 has a V-shaped cross section and a pointed distal end, with sharp leading edges 18. The aperture 15 may be a central V-shaped axial through slot configured to snugly receive the distal ends of the incision blade 5 and the correspondingly shaped safety shield 6, without impeding sliding extension and retraction of the safety shield 6.

The nose cone 4 may be comprised of polyethylene, polycarbonate, ABS, other plastic resin or other material suitable for surgical applications. Advantageously, the conical shape of the nose cone 4 facilitates dilation of an incision to the full instrument diameter, thus minimizing trauma to the penetrated tissue. Optionally, the nose cone 4 may be lubricated with a biocompatible surface treatment, such as by coating the surface of the nose cone 4 with one of the family of parylene compounds, such as those available from Specialty Coating Systems, Inc., Indianapolis, Ind. Parylene compounds comprise a family of p-xylylene dimers that polymerize when deposited onto a surface to form a hydrophobic polymeric coating. For example, a nose cone 4 according to principles of the invention may be coated with polymerized dichloro-(2,2)-paracyclophane (Parylene C) or di-p-xylylene (Parylene N). Parylene monomers may be applied to the surface of the nose cone 4 by gas-phase deposition in a vacuum chamber. The coating may further facilitate entry of the instrument into a body cavity.

The cartridge assembly 8 is received in the distal end of the sleeve tube/body member 2 as illustrated in FIG. 3A. The nose cone 4 interlocks with the spring retainer housing 23. A shoulder stop 16 provides an abutment which meets the distal end of the sleeve tube/body member 2 when the cartridge assembly 8 is installed. Male latches 17 provide mechanisms for engaging corresponding recesses 21 in the spring retainer housing 23. A heel pocket recess 30 receives heel stops 19 of the incision blade 5 to lock the incision blade 5 in position, as shown in FIG. 6.

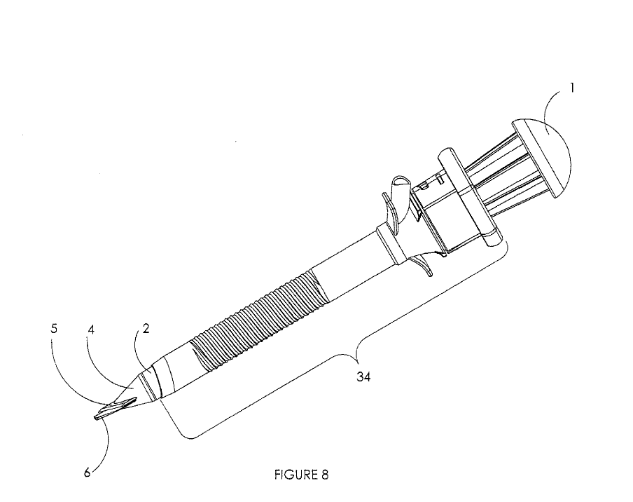

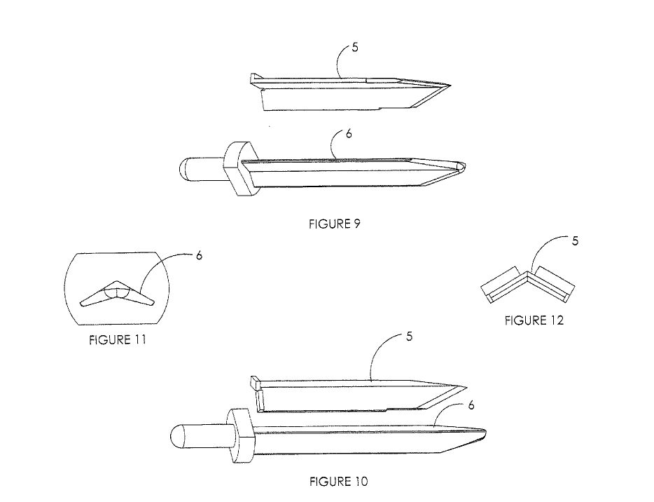

The pointed, double beveled edge 18 of the V-shaped incision blade 5 produces a clean incision with a slight flap. The pointed, double beveled edge 18 of the V-shaped incision blade 5 is also illustrated in FIG. 9. The V-shaped cross section for making a V-shaped incision is also clearly shown in FIGS. 10 and 12. With minimal force, the incision blade 5 produces a V-shaped incision without tearing or otherwise causing unnecessary trauma to the tissue. The incision with the flap, which is formed by the intersecting beveled edges, may be closed with relatively few stitches, staples or other closure means. The flap may also be folded during insertion of an instrument to provide a generally rectangular opening.

The incision blade 5 has at least two different widths. A narrow region 20 along the sides of the incision blade 5 ensures that the sides of the safety shield 6 extend beyond the sides of the incision blade 5 when the safety shield is deployed (i.e., extended). Thus, the shield prevents possible tissue or organ laceration while the device is inside a body cavity, even if a surgeon accidentally contacts an internal organ. Concomitantly, a wider region along the sides of the incision blade 5 at the aperture 15 of the nose cone 4 ensures a snug fit, such that the blade 5 will not shift from side to side within the aperture 15. Thus, the V-shaped incision blade is snugly positioned between the longitudal sides of the nose cones centrally located V-shaped axial through slot.

The safety shield 6 is configured to serve as a guard, seal and a surgical implement. The safety shield 6 may be made be comprised of plastic such as polyethylene, polycarbonate, ABS, other plastic resin or other material suitable for use in a surgical device. A blunt radius distal point 35, as shown in the side and head-on views of FIGS. 10 and 11, respectively, is provided to aid a surgeon in safely separating muscle fiber and to protect internal organs should accidental contact occur after the distal end of the device enters a body cavity. The safety shield 6 is configured to blend with the conical shape of the nose cone 4 when the shield 6 is fully retracted during a piercing procedure. The conforming streamlined contour of the safety shield 6 thus reduces resistance to penetration, tissue trauma during full instrument incision dilation and the risk of becoming plugged with loose tissue.

Furthermore, the safety shield is contoured to conform to the cross-sectional shape of the incision blade 5, as illustrated in FIGS. 4 and 9. Advantageously, the V-shape provides a thin geometry that reduces its drag or resistance against the abdominal wall tissue allowing rapid deployment through and immediately after a full width incision is produced by the V-shaped incision blade 5 but before the nose cone 4 has fully dilated the incision thereby offering nearly immediate internal organ protection.

Additionally, the safety shield 6 incorporates a forward travel stop 27 that maintains a maximum fully deployed blade shielded position. Upon penetrating a body cavity, resistance to the depressed safety shield 6 is relieved and the spring 5 causes the shield to rapidly deploy. Upon deployment, the travel stop 27 contacts the stop lug 26. The spring-driven impact of the travel stop 27 with the stop lug 26 produces a sensible signal. The signal may be tactile and/or audible and/or visible. Indeed the construction and composition of the lug 26 and stop 27 may be tailored to maximize detection of such signals.

The forward travel stop 27 serves as a spring 22 abutment seat. Protruding rearward from the forward travel stop 27 is a shield spring post stop 28 that provides for spring centering/alignment and a reward stop for full retraction. A contact point with a housing spring post stop 29 defines a point of retraction wherein the edges of the safety shield conform to the conical shape of the nose cones 4.

The spring retainer housing 23 is attached to the proximal end of the nose cone 4 during assembly by two sets of latching details, the latch/female 17 and the latch/male 21 interlock to one-another on opposing sides as illustrated in FIGS. 4 and 6. The latch/male 21 incorporates a standing stop lug 26 that abuts the back end of the nose cone 4 and heel stop 19 of the V-shaped incision blade 5, firmly securing and encapsulating the blade from movement within the heel pocket 30, as shown in FIG. 6. Additionally, as discussed above, the spring retainer housing 23 uses the standing stop lug 26 as a forward stop used by the V-shaped safety shield 6 to limit its travel and set the full deployment position. Furthermore, the latch/female 17 and the latch/male 21 interlocking is securely maintained when the assembly is installed into the sleeve tube/body member 2 due to the minimal clearance between the two components.

A housing spring post stop 29 provides a spring 22 centering/alignment standing post 29 for the spring proximal positioning and seat depth. The housing spring post stop 29 also provides a rearward contact stop for the V-shaped safety shields 6 spring post and stop 28. Upon contact of the spring post and stop 28 with the housing spring post stop 29, the V-shaped safety shield 6 is fully retracted thereby allowing the conical contours of the V-shaped safety shield 6 to blend with the contour of the nose cone 4. Referring now to FIGS. 6A and 6B, the assembled cartridge assembly 8 with the safety shield 6 in its extended deployed position is shown. In the top view of FIG. 6A, it is apparent that the deployed safety shield 6 extends beyond the cutting edges of the incision blade 5. Likewise, in the bottom view of FIG. 6B it is apparent that the cutting edge of the incision blade 5 does not extend beyond the deployed safety shield 6. Concomitantly, the head-on view of FIG. 6C shows the incision blade 5 conforming generally to the shape of the safety shield 6.

In sum, an incision blade/safety shield cartridge assembly 8 according to principles of the invention is comprised of a nose cone 4, a V-shaped incision blade 5, a V-shaped safety shield 6, a spring 22, and a safety shield spring retainer housing 23. A trocar according to principles of the invention is comprised of an incision blade/safety shield cartridge assembly 8, a sleeve tube/body member 2 and a handle 1. Assembly of the incision blade/safety shield cartridge assembly 8 entails inserting the internal V-shaped incision blade 5 and safety shield 6 into nose cone 4, placing the spring 22 between the safety shield spring centering/locating post 29 and the stop lug 26, positioning the forward travel stop 27 of the safety shield 6 between the distal end of the spring 22 and the stop lug 26, and coupling (e.g., via snap locks) the nose cone 4 to the safety shield spring retainer housing 23. In this manner, the cartridge is assembled without need for bonding or other attachment means. The assembled incision blade/safety shield cartridge assembly 8 may then be mated to the tube 2 of a trocar according to principles of the invention, or any other trocar configured to mate with the cartridge assembly 8.

The handle 1 is preferably comprised of plastic of polycarbonate, ABS or other plastic resin. The curved or mushroom shape fits a surgeon's palm for comfort and control. Handle ribs 24 ensure structural integrity while enabling mass/weight reduction for a substantial surgeon gripping diameter or size. The handle may be bonded or otherwise mechanically secured to the proximal end of the sleeve tube/body member.

The sleeve tube/body member 2 may be made from thin wall stainless steel tubing of polished or mat finish or of polycarbonate, ABS or other plastic resin. The blunt end or distal end, may receive the incision blade/safety shield cartridge assembly 8 to its full predetermined depth of the shoulder stop detail 16. This blunt end may be square cut allowing a nearly gap free mating when the shoulder stop 16 surface abuts to the tube 2 during final assembly. Furthermore, the outside diameter of the portion of the incision blade/safety shield cartridge assembly 8, which fits within the inside diameter of the sleeve tube/body member, has minimal clearance, thereby resulting in nearly mismatch free matching of the largest outside diameter of the nose cone 4 and the outside diameter of the sleeve tube/body member 2.

A V-shaped indented staking detail 3 may be used for fixed retention of the internal incision blade/safety-shield cartridge assembly 8. One or more other mechanical attachments may be provided to secure the cartridge assembly 8 at multiple locations about the periphery of the tube 2. In an exemplary implementation, the point of the “V” of the staking detail 3 faces the distal end of the trocar. The point is pierced first, through the sleeve tube/body member and continues piercing and bending a V-shaped tab of metal downward and rearward into the receiving area designed for the V-shaped staking retention. This downward and rearward motion draws rearward and secures the incision blade/safety shield cartridge assembly, ensuring abutment of the distal, square cut end, of the sleeve tube/body member 2 and the mating abutment shoulder stop of the nose cone 4.

The exemplary incision blade 5 of a trocar according to principles of the invention offers numerous advantages over the prior art. A V-shaped blade cutting contour that tapers to a pointed tip, improves incision and penetration capability by allowing for a small V-shaped incision which facilitates entry of the trocar body and a cannula. The incision blade 5 is preferably a V-shaped blade with surgical cutting edges which allows for a clean and precise V-shaped incision. The V-shaped incision consequently minimizes tearing of surrounding tissue when a cannula or other surgical instrument is inserted. This provides an important advantage over prior art blades with a single planar cutting edge that is conducive to tearing, and over prior art blades with three or more cutting edges that cause unnecessary trauma and increased risk of collateral injury. The blade design of a trocar according to principles of the invention reduces risk of collateral damage beacuse the V-shaped incision readily allows passage of the safety shield 6 and a cannula. This is due to the fact that the blade cutting contour of a trocar according to principles of the invention produces an incision with a foldable flap to allow better access to a body cavity with less unnecessary tearing and injury within the location being cut and surrounding area. While a V-shaped blade cutting contour is preferred, other flap forming configurations, such as L-shaped, U-shaped, W-shaped and the like also come within the scope of the invention.

Disadvantageously, multiple cutting edges in multiple planes, as known in the prior art, may not only cause unavoidable tearing of adjacent internal organs and muscle but also may cut unnecessarily large incisions during, laparoscopic procedures. This may result in, for example, pressurized carbon dioxide gas escaping from the area being treated. As insufflation is necessary for many laparoscopic procedures, the large incisions and loss of gas may result in the need for additional gas to be pumped into the internal area being treated or may result in the need for seals to be placed around the cannula in order to maintain proper insufflation. The novel cutting contour of the incision blade 5 of a trocar according to principles of the invention cuts a more precise and resilient incision that avoids the evacuation of pressurized gas which may occur during use of prior art devices. By better maintaining the integrity of insufflation, a trocar according to principles of the invention may reduce both the length of surgery and related costs.

Furthermore, the cutting contour of a trocar according to principles of the invention may be tapered to a degree of thinness not possible in, for example, a pyramidal configuration as is common in the prior art. In the assembly of a trocar according to principles of the invention, the incision blade cutting angles may be customized to suit particular locations on a body to be cut, particular procedures, and a surgeon's particular needs and preferences.

Of course, the incision blade 5 and the V-shaped blade cutting contour, as detailed above, could be applied to numerous trocars as are known in the art. Such other applications are intended to come within the scope of the invention.

The simplicity of the incision blade 5 and safety shield 6 of a trocar according to principles of the invention not only leads to greater reliability and increased safety, but it also improves the utility of a trocar. A cartridge assembly 8 according to principles of the invention has been intentionally designed to permit use with a variety of trocars and similar surgical tools without compromising its effectiveness. Additionally, those skilled in the art will appreciate that any compatible cannula may be employed with a trocar according to principles of the invention. The invention is not limited to any particular type of cannula.

The exemplary trocar may be utilized in surgery to provide a relatively small access opening through outer tissue and muscle layers into an internal body cavity. The cavity may be insufflated by the introduction of gas prior to use of the trocar. During a laparoscopic procedure, the trocar is coaxially aligned with the cannula. The distal end of the trocar is forced into a determined area of the body cavity. A V-shaped safety shield 6 is biased to initially extend beyond the piercing tip of the V-shaped incision blade 5. As the shield 6 is forced against the body cavity tissue to be pierced, the shield retracts into the nose cone 4 and sleeve tube 2 member. When the body cavity is breached, a spring biasing member returns the blunt end of the safety shield 6 past the piercing tip of the incision blade 5 to prevent accidental puncturing or laceration of the internal organs by the sharpened point. Because the safety shield 6 is contoured After the trocar produces an access opening, the trocar is removed and the cannula may be left secured in place in the opening. Thus, the cannula provides an open conduit into the body cavity.

While an exemplary embodiment of the invention has been described in detail, it should be apparent that modifications and variations thereto are possible, all of which fall within the true spirit and scope of the invention. With respect to the above description then, it is to be realized that the optimum relationships for the components of the invention and steps of the process, to include variations in form, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention.

What is claimed is:

1. An incision blade/safety shield cartridge assembly comprised of a nose cone having an aperture, a stationary V-shaped incision blade having a distal end extending from said aperture, a spring-biased V-shaped safety shield conformed to the shape of the stationary V-shaped incision blade and movable from an extended position extending beyond the distal end of said stationary V-shaped incision blade to a retracted position revealing the distal end of said stationary V-shaped incision blade, said V-shaped safety shield having a distal end and a proximal end, a spring adapted to bias the spring-biased V-shaped safety shield, and a safety shield spring retainer housing operably coupled to said nose cone and configured to support the spring against the proximal end of the spring-biased V-shaped safety shield.

2. An incision blade/safety shield cartridge assembly according to claim 1, wherein said spring-biased V-shaped safety shield includes a travel stop, and said safety shield spring retainer housing includes a stop lug adapted to define an abutment for the travel stop when the spring-biased V-shaped safety shield is biased to the extended position.

3. An incision blade/safety shield cartridge assembly according to claim 2, wherein said travel stop and stop lug are adapted to produce a sensible signal when the spring-biased V-shaped safety shield is biased to the extended position.

4. An incision blade/safety shield cartridge assembly according to claim 3, wherein the sensible signal includes at least one signal from the group consisting of a tactile signal, an audible signal and a visible signal.

5. An incision blade/safety shield cartridge assembly according to claim 1, wherein said nose cone includes a biocompatible lubricated surface treatment.

6. An incision blade/safety shield cartridge assembly according to claim 1, wherein said V-shaped incision blade is adapted to produce a clean v-shaped incision in a body cavity.

7. An incision blade/safety shield cartridge assembly according to claim 1, wherein said V-shaped incision blade includes a pointed double-beveled edge at its distal end.

8. An incision blade/safety shield cartridge assembly according to claim 1, wherein said V-shaped safety shield is adapted to retract to the retracted position during piercing of a body cavity and rapidly extend to the extended position upon penetration of a body cavity.

9. A surgical instrument comprised of an incision blade/safety shield cartridge assembly, a sleeve body having a distal end and an opposite proximal end, and a handle, said incision blade/safety shield cartridge assembly being coupled to the sleeve body at the distal end, said handle being coupled to the sleeve body at the proximal end, said incision blade/safety shield cartridge assembly being comprised of a nose cone having an aperture, a stationary V-shaped incision blade having a distal end extending from said aperture, a spring-biased V-shaped safety shield conformed to the shape of the stationary V-shaped incision blade and movable from an extended position extending beyond the distal end of said stationary V-shaped incision blade to a retracted position revealing the distal end of said stationary V-shaped incision blade, said V-shaped safety shield having a distal end and a proximal end, a spring adapted to bias the spring-biased V-shaped safety shield, and a safety shield spring retainer housing operably coupled to said nose cone and configured to support the spring against the proximal end of the spring-biased V-shaped safety shield.

10. A surgical instrument according to claim 9, wherein said handle has a mushroom shape.

11. A surgical instrument according to claim 9, wherein said nose cone includes a biocompatible lubricated surface treatment.

12. A surgical instrument according to claim 9, wherein said V-shaped incision blade is adapted to produce a clean v-shaped incision.

13. A surgical instrument according to claim 9, wherein said V-shaped incision blade includes a pointed double-beveled edge at its distal end.

14. A surgical instrument according to claim 9, wherein said V-shaped safety shield is adapted to retract to the retracted position during piercing of a body cavity and rapidly extend to the extended position upon penetration of a body cavity.

15. A surgical instrument according to claim 9, wherein said spring-biased V-shaped safety shield includes a travel stop, and said safety shield spring retainer housing includes a stop lug, said stop lug adapted to define an abutment for the travel stop when the spring-biased V-shaped safety shield is biased to the extended position.

16. A surgical instrument according to claim 15, wherein said travel stop and stop lug are adapted to produce a sensible signal when the spring-biased V-shaped safety shield is biased to the extended position.

17. A surgical instrument according to claim 16, wherein the sensible signal includes at least one signal from the group consisting of a tactile signal, an audible signal and a visible signal.

18. A surgical instrument comprised of an incision blade/safety shield cartridge assembly, a sleeve body having a distal end and an opposite proximal end, and a handle, said incision blade/safety shield cartridge assembly being coupled to the sleeve body at the distal end, said handle being coupled to the sleeve body at the proximal end, said incision blade/safety shield cartridge assembly being comprised of a nose cone having an aperture, a stationary incision blade having a distal end extending from said aperture, a spring-biased safety shield conformed to the shape of the stationary incision blade and movable from an extended position extending beyond the distal end of said stationary incision blade to a retracted position revealing the distal end of said stationary incision blade, said safety shield having a distal end and a proximal end, a spring adapted to bias the spring-biased safety shield, and a safety shield spring retainer housing operably coupled to said nose cone and configured to support the spring against the proximal end of the spring-biased safety shield, said safety shield being adapted to retract to the retracted position during piercing of a body cavity and rapidly extend to the extended position upon penetration of a body cavity.

19. A surgical instrument according to claim 18, wherein said incision blade includes a pointed double-beveled edge at its distal end is adapted to produce a clean incision with a flap.

20. A surgical instrument according to claim 9, wherein said spring-biased safety shield includes a travel stop, and said safety shield spring retainer housing includes a stop lug, said stop lug adapted to define an abutment for the travel stop when the spring-biased safety shield is biased to the extended position, said travel stop and stop lug being adapted to produce a sensible signal when the spring-biased safety shield is biased to the extended position, said sensible signal including at least one signal from the group consisting of a tactile signal, an audible signal and a visible signal.
